(19) United States
(12) Patent Application Publication
(10) Pub. No.: US 2026/0199610 A1
(43) Pub. Date: Jul. 16, 2026

(54) INSULIN PEN-TYPE SYRINGE REFRIGERATION CUP

(71) Applicant: Zhejiang Sunclose Technology CO., LTD., Jinhua (CN)

(72) Inventors: HUAJIAN ZHANG, Jinhua (CN); JINGPING LI, Jinhua (CN)

(21) Appl. No.: 19/371,606

(22) Filed: Oct. 28, 2025

(30) Foreign Application Priority Data
Jan. 15, 2025 (CN) 202520089178.1

(51) Int. Cl. A61M 5/44; A61M 5/00

(52) U.S. Cl. CPC A61M 5/44; A61M 5/002; A61M 2205/3606; A61M 2205/362; A61M 2205/3633; A61M 2205/3673; A61M 2205/8206

(57) ABSTRACT

An insulin pen-type syringe refrigeration cup is provided, which is provided with an insulin pen cartridge. The insulin pen-type syringe refrigeration cup includes a refrigeration component, a cold conduction tube, a cup lid, and a cup body. The cold conduction tube defines a storage space that accommodates insulin pens, the storage space is also suitable for accommodating the insulin pen cartridge. A diameter of the cold conduction tube is 20 mm-50 mm. The cold conduction tube includes an avoidance groove. The cup lid is suitable for accommodating the refrigeration component. The cup body includes a cup wall and an insulation chamber. The cup lid can detachably seal the insulation chamber, and the refrigeration component keeps the insulation chamber in a low temperature state. The present disclosure significantly reduces the volume by limiting the size of the cold conduction tube and providing the avoidance groove.

### CROSS-REFERENCE TO RELATED APPLICATIONS

This application claims priority to Chinese Patent Application No. 202520089178.1, filed on January 15, 2025, which is hereby incorporated by reference in its entirety.

### TECHNICAL FIELD

The present disclosure relates to the field of medical device technologies, and in particular, to an insulin pen-type syringe refrigeration cup.

### BACKGROUND

Diabetes is a common endocrine metabolic disease; patients need to use for insulin self-injection and oral hypoglycemic drugs. The use of insulin has certain temperature requirements and must be stored in a low-temperature environment. For users with travel needs, existing storage devices have many inconveniences. Currently, most insulated cups on the market are designed to store multiple insulin pen cartridges, and insulin pen-type syringes, all called as, insulin pens, which need to be stored separately. Besides that, there is a type of insulated medicine box that can simultaneously store insulin pens and insulin pen cartridges. However, the prominent problem faced by both types of products is their large size and lack of portability. Whether it is traveling, business trips, or short daily outings, a larger size can cause inconvenience to users and increase their carrying burden. Furthermore, a pen clip of insulin pens usually protrudes from a pen body, thereby occupying additional space, resulting in an increase in the size of the cold conduction tube and cup body. At the same time, some equipment that uses foam as thermal insulation material has poor thermal insulation effect. In order to achieve a certain heat preservation duration, the thickness of foam insulation layer must be increased, which not only further increases the overall volume, but also makes the equipment appear bulky, and the foam material itself has limited protective performance, which cannot provide a reliable storage environment for insulin.

### SUMMARY

The purpose of the present disclosure is to provide an insulin pen-type syringe refrigeration cup that can simultaneously store insulin pens and insulin pen cartridges, significantly reduce volume, improve portability, and facilitate users to easily carry them in various travel scenarios and meet storage requirements.

To achieve the above objectives, the present disclosure provides an insulin pen-type syringe refrigeration cup, where the insulin pen is provided with an insulin pen cartridge, also known as insulin injection solution; and the insulin pen-type syringe refrigeration cup includes:

a refrigeration component, which includes a semiconductor refrigeration chip, a heat conduction component, and a heat dissipation fan; where the semiconductor refrigeration chip includes a cold end and a hot end, and the hot end is in contact with the heat conduction component,

a cold conduction tube, which includes a sealed end and an open end, where the cold end is in contact with the sealed end, the cold conduction tube includes an avoidance groove extending from the open end to the sealed end, where the cold conduction tube defines a storage space that is configured to accommodate up to one or two insulin pens, and the storage space is further configured to accommodate the insulin pen cartridge, and/or, a diameter of the cold conduction tube is 20 mm to 50 mm,

a cup lid, configured to accommodate the refrigeration component, where the sealed end is fixedly connected to the semiconductor refrigeration chip and/or the cup lid,

a cup body, which includes a cup wall and an insulation chamber enclosed by the cup wall, where the open end extends into the insulation chamber, the cup lid is configured to detachably seal the insulation chamber, and a part of the insulin pen or a part of the insulin pen cartridge is configured to extend into the avoidance groove or enter the insulation chamber by passing through the avoidance groove; which improves space utilization, rendering the placement of the insulin pen in the refrigerated cup more reasonable, and the refrigeration component keeps the insulation chamber at a low temperature.

In some embodiments of the present disclosure, the diameter of the cold conduction tube refers to an inner diameter of the cold conduction tube, the cold conduction tube has a thickness of 1 mm. The setting of the avoidance groove can also use some space occupied by the cold conduction tube and some inner space of the insulation chamber to accommodate the insulin pen or the insulin pen cartridge.

In some embodiments of the present disclosure, two insulin pens are configured to be inserted side by side into the cold conduction tube through the open end, or the insulin pen and the insulin pen cartridge are configured to be inserted side by side into the cold conduction tube through the open end. This design helps to shorten lengths of the cold conduction tube and cup body, thereby rendering the structure more compact and portable.

In some embodiments of the present disclosure, the cup wall includes an inner wall, an outer wall, and a hollow cavity provided between the two, the hollow cavity is in a vacuum state, and there is an insulation gap between the cold conduction tube and the inner wall. The insulin pen can be extended into the insulation gap. Compared to traditional insulation materials, vacuum insulation structures are more lightweight and meet the positioning of portable products.

In some embodiments of the present disclosure, the cold conduction tube includes two avoidance grooves that are symmetrically arranged, allowing a user to easily find the corresponding avoidance grooves no matter which direction he inserts the insulin pen from, thereby improving the convenience and fault tolerance of operation.

In some embodiments of the present disclosure, lengths of the avoidance grooves do not exceed 40% of a length of the cold conduction tube; and/or widths of the avoidance grooves do not exceed 15% of a perimeter of the cold conduction tube. This limitation can meet the requirement of a pen clip entering the insulation gap through the avoidance grooves, ensuring the structural integrity of the cold conduction tube, maintaining good cooling performance of the cold conduction tube, and ensuring a low temperature environment inside the insulation chamber.

In some embodiments of the present disclosure, the avoidance grooves include a main body groove, and an open groove opening and a sealed groove bottom that are provided at two ends of the main body groove, the open groove opening is provided at the open end, which enables the insulin pen clip to easily align and insert into the avoidance grooves; the open groove structure plays a guiding role, providing clear insertion direction and position indication for the user, reducing the difficulty of inserting the pen clip, and improving the convenience and efficiency of operation. The sealed groove bottom is in a circular arc structure, which prevents the pen clip from being damaged due to collision with the sharp groove bottom during insertion and provides protection for the pen clip.

In some embodiments of the present disclosure, the insulin pen-type syringe refrigeration cup further includes a power supply component configured to supply energy to the refrigeration component; the cup lid is provided with a first chamber and a second chamber that are arranged in parallel; the first chamber is configured to accommodate the refrigeration component, and the second chamber is configured to accommodate a refrigeration control circuit board; the power supply component is arranged below the second chamber, and the refrigeration control circuit board is electrically connected to the refrigeration component and the power supply component, respectively.

An interior of the cup lid is reasonably planned into different chambers to accommodate various components, and the precise spatial layout fully utilizes the limited space inside the cup lid, further compressing the overall volume of the device and enabling the integration of multiple functions within the limited space. There is no need to install the refrigeration component and control circuit board separately at different positions on the cup body, and then perform tedious circuit connections, greatly simplifying the production assembly process, improving production efficiency, and reducing the risk of possible failures due to complex assembly.

In some embodiments of the present disclosure, the semiconductor refrigeration chip is horizontally arranged in the first chamber and separates the first chamber to form a heat conduction chamber and a cold conduction chamber, the heat conduction chamber is configured to accommodate the heat conduction fan and the heat conduction component, the cup lid is provided with an air inlet and an air outlet that are configured to communicate the heat conduction chamber, the sealed end is configured to extend into the cold conduction chamber, and the cup body extends into the cold conduction chamber and is detachably connected to the cup lid.

In some embodiments of the present disclosure, the first chamber is cylindrical in shape, the cold conduction chamber is provided with an internal threaded structure, the cup body is provided with an external threaded structure, and the cup body is connected to the cup lid by threads.

In some embodiments of the present disclosure, the heat dissipation fan includes a fan body and a fan frame configured to install the fan body, and the fan frame is clamped to the cup lid.

In some embodiments of the present disclosure, the power supply component includes a battery housing, the battery housing includes an annular shell that matches a shape of the cup body, and the power supply component and the cup body jointly support the insulin pen-type syringe refrigeration cup. Due to the shape adaptation between the annular shell and the cup body, gaps or empty areas caused by shape mismatch are avoided, allowing the power supply component to cleverly integrate into the overall structure and achieve higher integration. Specifically, the cup body includes a first bottom surface, and the battery housing includes a second bottom surface. The first bottom surface and the second bottom surface jointly support the insulin refrigeration cup, making it more stable to place. The first chamber is communicated to the second chamber for easy wiring.

In some embodiments of the present disclosure, an anti-slip pad is provided on one side of the battery housing away from the cup body. The anti-slip pad is a vertical plane, thereby providing a more stable surface for the user to apply lateral force with his fingers, preventing the cup from rotating or sliding in his hands, rendering one handed operation easier and more accurate.

In some embodiments of the present disclosure, the power supply component includes a rechargeable battery and a power control circuit board arranged inside the battery housing. The power control circuit board is provided with a charging interface embedded in the battery housing, and the battery housing is detachably connected to the cup lid. The power control circuit board is electrically connected to the refrigeration control circuit board.

In some embodiments of the present disclosure, the cup lid is embedded with an operation button and a display unit, both of the operation button and the display unit are electrically connected to the refrigeration control circuit board. The operation button inputs a control instruction to the refrigeration control circuit board to control the refrigeration component. The display unit is suitable for providing refrigeration temperature and battery power, rendering it convenient for the user to operate and real-time understand the operating state of the device, and thereby improving the user experience.

The beneficial effects of the present disclosure are as follows.

By providing the cold conduction tube that can accommodate up to one insulin pen and one insulin pen cartridge, the overall structure is more compact, significantly reducing the volume of the insulin refrigeration cup and meeting the portability needs, rendering it easy for the user to carry in various travel scenarios. On the other hand, it can more accurately and quickly adjust the internal temperature to achieve and maintain the required low temperature state, and the volume of the power supply component that supplies energy to the refrigeration component can also be reduced accordingly, thus achieving an overall lightweight design.

Furthermore, by providing the avoidance grooves to store the pen clip into the insulation gap, the placement of the insulin pen in the cold conduction tube is more compact, shortening the widths of the cold conduction tube and cup body, and improving the utilization of internal space of the device. The setting of the avoidance grooves at the same time provides a clear insertion path for the pen clip, thereby better fixing the position of the insulin pen, preventing or reducing shaking in the cold conduction tube, and ensuring the normal use of the insulin pen and the quality and safety of insulin.

Compared with foam insulation, vacuum insulation has better effect and is lighter. It can effectively maintain low temperature environment without heavy insulation layer, avoid volume increase due to insulation structure, and realize compact design.

### BRIEF DESCRIPTION OF DRAWINGS

FIG. 1 is a schematic structural diagram of an insulin pen-type syringe refrigeration cup provided by the present disclosure.

FIG. 2 is an exploded schematic diagram of the insulin pen-type syringe refrigeration cup provided by the present disclosure.

FIG. 3 is a schematic sectional view of the insulin pen-type syringe refrigeration cup provided by the present disclosure.

FIG. 4 is an exploded schematic diagram of a cup lid and components integrated on the cup lid provided by the present disclosure.

FIG. 5 is a schematic sectional view of the cup lid provided by the present disclosure.

Numeral reference: cup lid 100, first chamber 101, second chamber 102, heat conduction chamber 103, cold conduction chamber 104, air outlet 105, air inlet 106, cup body 200, cup wall 201, insulation chamber 202, first bottom surface 203, power supply component 300, battery housing 301, battery 302, power control circuit board 303, annular shell 304, anti-slip pad 305, second bottom surface 306, charging interface 311, semiconductor refrigeration chip 401, heat conduction component 402, heat dissipation fan 403, fan body 404, fan frame 405, cold conduction tube 500, sealed end 501, open end 502, avoidance groove 503, main body groove 504, open groove opening 505, sealed groove bottom 506, refrigeration control circuit board 601, operation button 602, display unit 603, stylus 604.

### DESCRIPTION OF EMBODIMENTS

In order to clarify the purpose, technical solution, and advantages of the present disclosure, a detailed description of the present disclosure will be provided below in combination with the accompanying drawings and specific embodiments.

Here, it should be noted that in order to avoid blurring the present disclosure due to unnecessary details, only structures and/or processing steps closely related to the solution of the present disclosure are shown in the accompanying drawings, and other details that are not closely related to the present disclosure are omitted.

Besides that, it should be noted that terms “including”, “containing”, or any other variation thereof are intended to encompass nonexclusive inclusion, such that a process, method, item, or device that includes a series of elements not only includes those elements, but further includes other elements that are not explicitly listed or are inherent to such a process, method, item, or device.

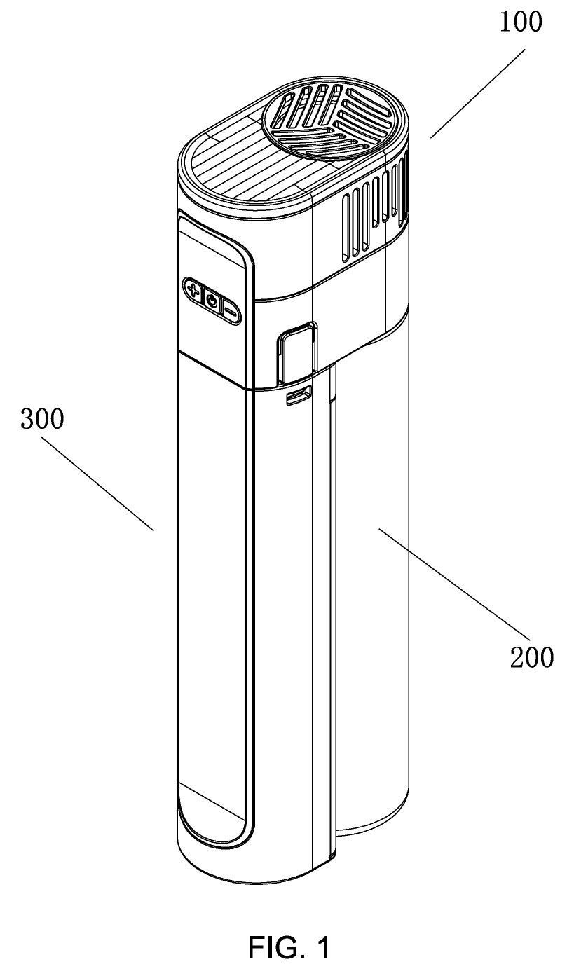

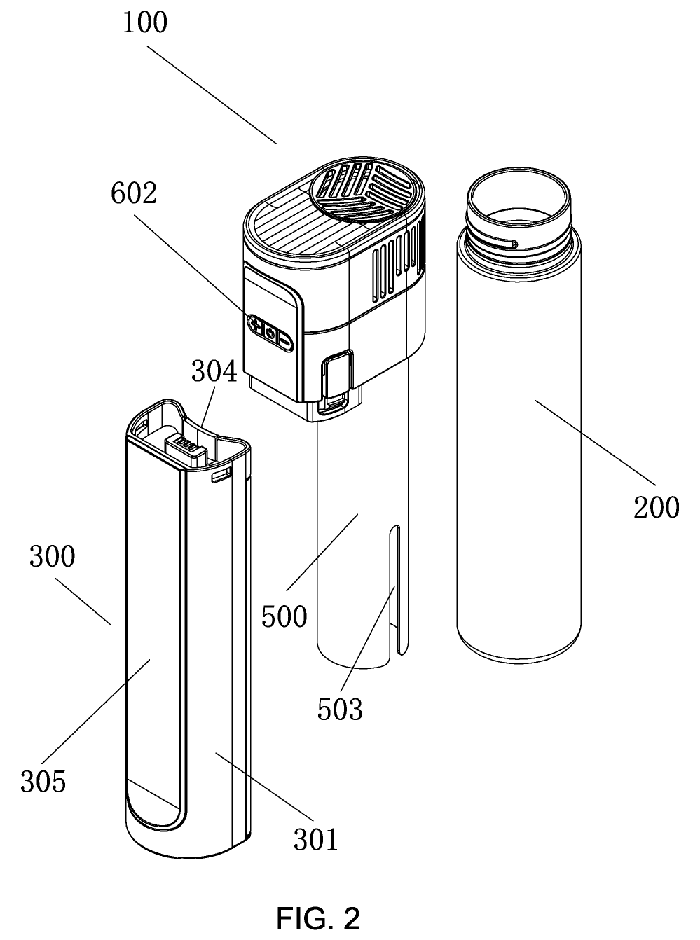

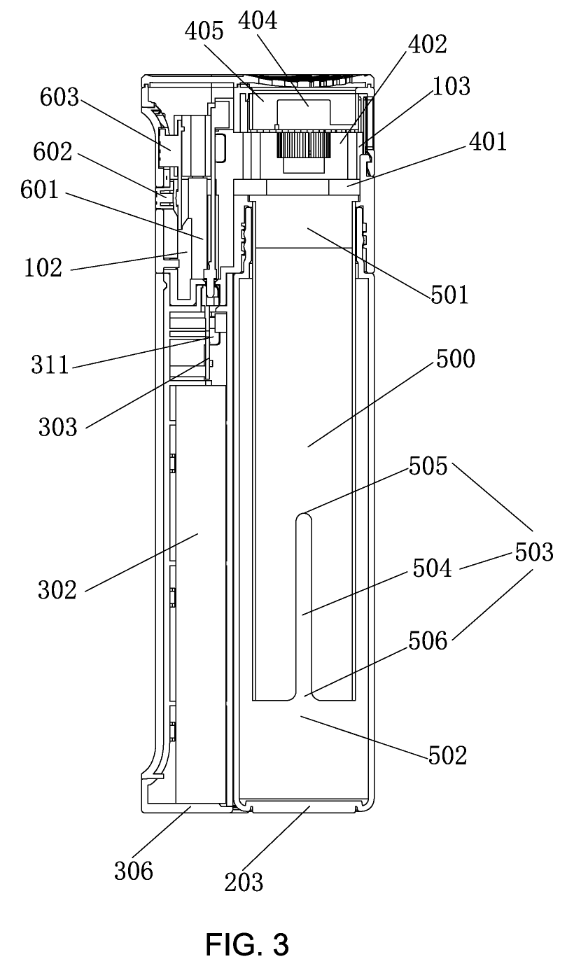

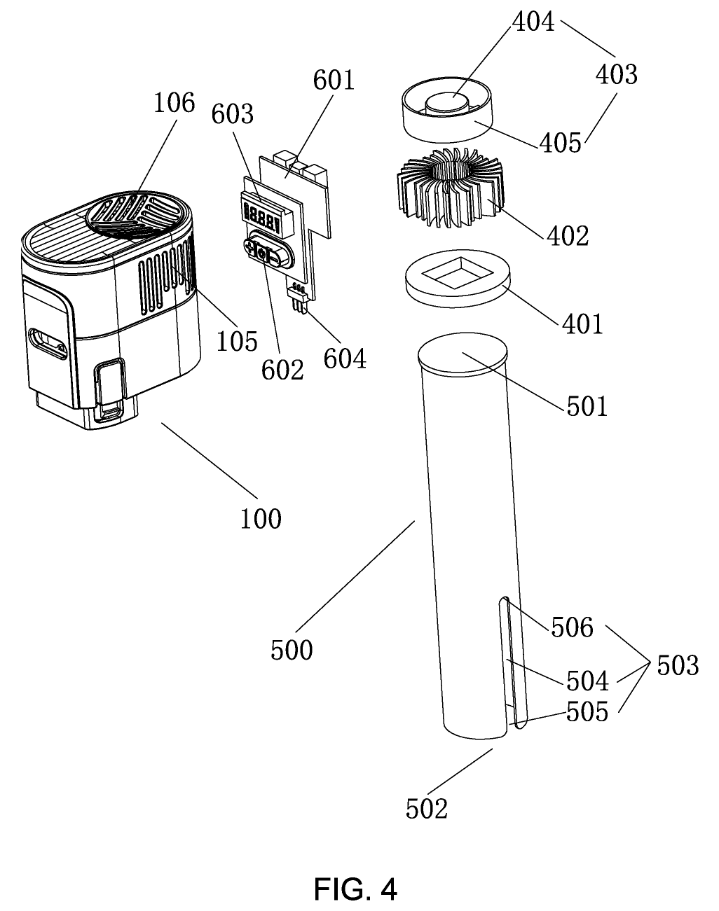

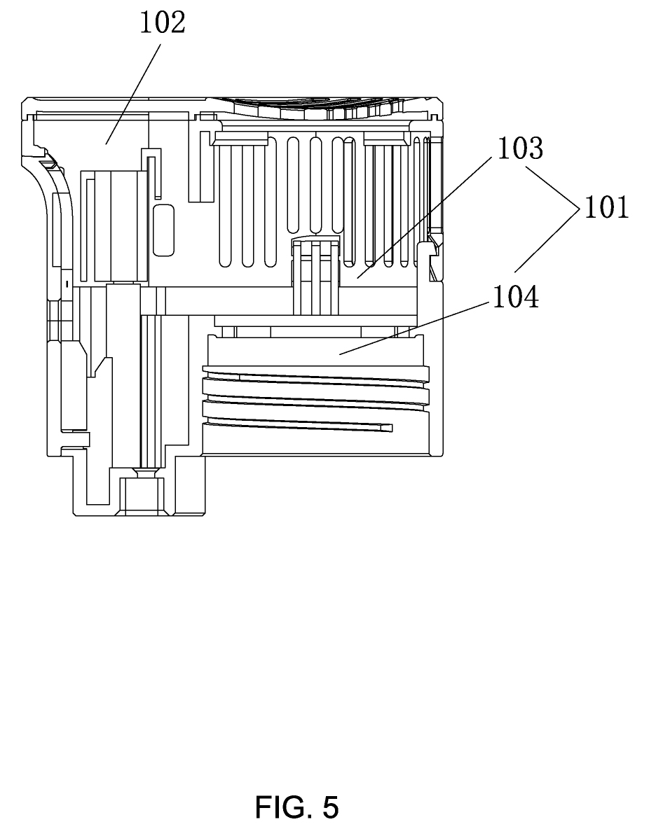

As shown in FIGS. 1-5, an insulin pen-type syringe refrigeration cup includes a cup lid 100, a cup body 200, and a power supply component 300. The cup lid 100 integrates a refrigeration component, a cold conduction tube 500, a refrigeration control circuit board 601, an operation button 602, and a display unit 603. The cup body 200 includes a cup wall 201 and an insulation chamber 202 enclosed by the cup wall 201. The cup lid 100 is provided with a first chamber 101 and a second chamber 102 arranged in parallel. The first chamber 101 is suitable for accommodating the refrigeration component, and the first chamber 101 is cylindrical. The semiconductor refrigeration chip 401 is horizontally arranged in the first chamber 101 and separates the first chamber 101 to form a heat conduction chamber 103 and a cold conduction chamber 104. The heat conduction chamber 103 is suitable for accommodating a heat dissipation fan 402 and a heat conduction component 402. The cup lid 100 is formed with an air inlet 106 and an air outlet 105 suitable for communicating the heat conduction chamber 103. In an implementation mode, the semiconductor refrigeration chip 401 includes a cold end and a hot end, the hot end is connected to the heat conduction component 402, i.e., the semiconductor refrigeration chip 401 supports the heat conduction component 402. The heat dissipation fan includes a fan body 404 and a fan frame 405 suitable for installing the fan body 404. The fan frame 405 is clamped to the cup lid 100. The cold conduction tube 500 includes a sealed end 501 and an open end 502. The sealed end 501 is adapted to extend into the cold conduction chamber 104 and adhere to the cold end. The sealed end 501 is fixedly connected to the semiconductor refrigeration chip 401. The open end 502 extends into the insulation chamber 202, and the cup lid 100 can detachably seal the insulation chamber 202. The refrigeration component keeps the insulation chamber 202 at a low temperature. In an implementation mode, the cold conduction chamber 104 is provided with an internal threaded structure, and the cup body 200 is provided with an external threaded structure. The cup body 200 is connected to the cup lid 100 by threads. At most one insulin pen and one insulin pen cartridge are inserted side by side into the cold conduction tube 500 through the open end 502. The insulin pen is provided with the insulin pen cartridge, also known as insulin injection. The cold conduction tube 500 defines a storage space that can accommodate up to one or two insulin pens, and the storage space is also suitable for accommodating the insulin pen cartridge. A diameter (inner diameter) of the cold conduction tube 500 is 40 mm, and two insulin pens can be inserted side by side into the cold conduction tube 500 from the open end 502. Insulin pens and insulin pen cartridges are also suitable for insertion into the cold conduction tube 500 through the open end 502 side by side. It can be understood that the diameter of the insulin pen cartridge is smaller than that of the insulin pen, and a length of the insulin pen cartridge is also smaller than that of the insulin pen. Two to three insulin pen cartridges can be arranged end-to-end in the cold conduction tube 500 or the insulation chamber 202. The cold conduction tube 500 includes two avoidance grooves 503 that are symmetrically arranged, and the two avoidance grooves 503 extend from the open end 502 to the sealed end. Both insulin pens and insulin pen cartridges are generally irregular bodies, and a part of the insulin pens or a part of the insulin pen cartridges can enter the insulation chamber 202 through the avoidance grooves. The insulin pens or insulin pen cartridges can be placed more closely with the cold conduction tube 500, thereby improving space utilization and better fixing the position of the insulin pens or insulin pen cartridges, preventing or reducing their shaking inside the cold conduction tube 500. A thickness of the cold conduction tube 500 is 1 mm, and the setting of the avoidance grooves 503 can also use some space occupied by the cold conduction tube 500 and some space inside the insulation chamber 202 to accommodate the insulin pens or the insulin pen cartridges.

In this embodiment, a volume of the heat preservation chamber 202 or the storage space is 200 ml, and an insulin pen with insulin pen cartridge and a spare insulin pen cartridge can meet the needs of patients with severe diabetes for half a month or patients with mild diabetes for one month, and can fully meet the needs of short trips or business trips.

It should be clarified that the positions of the air inlet 106 and the air outlet 105 are flexible. One feasible way is to arrange the air inlet 106 on an end face of the cup lid 100 and the air outlet 105 on a circumference of the cup lid 100. Of course, an opposite arrangement can also be adopted, that is, the air inlet 106 is provided on the circumference of the cup lid 100, and the air outlet 105 is provided on the end face of the cup lid 100. No matter what setting method is adopted, it should be considered within the protection scope of this application, and the protection scope of this application should not be limited by the specific setting positions of the air inlet 106 and the air outlet 105. This design aims to provide multiple feasible solutions for achieving effective communication and heat exchange between the cup lid and the heat conduction chamber so as to meet different usage scenarios and design requirements.

In this embodiment, lengths and widths of the avoidance groove 503 allow a pen clip to enter and exit at least. The lengths of the avoidance grooves 503 shall not exceed 40% of a length of the cold conduction tube 500, and the widths of the avoidance groove 503 shall not exceed 15% of a perimeter of the cold conduction tube 500. The main function of the cold conduction tube 500 is to transfer cold generated by the semiconductor refrigeration chip 401 to the insulin pens and the insulin pen cartridges. The size of the avoidance grooves 503 is limited to ensure that the cold conduction tube 500 has sufficient effective area to contact and transfer the cold to the insulin pens and insulin pen cartridges, so that insulin can be stored in a suitable low-temperature environment. As mentioned above, in an implementation mode, the size of the avoidance grooves 503 is larger, allowing more space inside the insulation chamber 202 to be utilized and improving storage capacity, but the refrigeration effect is correspondingly degraded. The avoidance grooves 503 include a main body groove 504, and an open groove opening 505 and a sealed groove bottom 506 located at two ends of the main body groove 504. The open groove opening 505 is formed at the open end 502, allowing the insulin pens or insulin pen cartridges to be easily aligned and inserted into the avoidance grooves 503. The open groove opening 505 has excessive rounded corners to prevent scratching the insulin pens or the insulin pen cartridges.

In this embodiment, the cup wall 201 includes an inner wall, an outer wall, and a hollow cavity (not shown in the figure) provided between the two, and the hollow cavity is in a vacuum state. There is an insulation gap between the cold conduction tube 500 and the inner wall. Compared to traditional insulation materials, vacuum insulation structures are lightweight and meet the positioning of portable products.

In this embodiment, the second chamber 102 accommodates the refrigeration control circuit board 601. The operation button 602 and the display unit 603 are embedded in the cup lid 100 adjacent to the second chamber 102, both operation button 602 and the display unit 603 are electrically connected to the refrigeration control circuit board 601. The operation button 602 inputs a control instruction to the refrigeration control circuit board 601 to control the refrigeration component. The display unit 603 is suitable for providing refrigeration temperature and battery power of the battery 302, facilitating user operation and real-time understanding of the operating state of the device, improving the user experience. The power supply component 300 is provided below the second chamber 102, and the refrigeration control circuit board 601 is electrically connected to both the refrigeration component and the power supply component 300. The first chamber 101 is communicated to the second chamber 102 for easy wiring, and the power supply component 300 is suitable for supplying power to the refrigeration component and the display unit 603. The refrigeration component includes the semiconductor refrigeration chip 401, a heat conduction component 402, and the heat dissipation fan 403.

In this embodiment, the power supply component 300 includes a battery housing 301, the rechargeable battery 302 arranged inside the battery housing 301, and the power control circuit board 303. The power control circuit board 303 is provided with a charging interface 311, and the charging interface 311 is embedded in the battery housing 301. The battery housing 301 is detachably connected to the cup lid 100, the power control circuit board 303 is in pressure contact with the refrigeration control circuit board 601. The battery housing 301 includes an annular shell 304 that matches a shape of the cup body 200, and the power supply component 300 and the cup body 200 jointly support the insulin pen-type syringe refrigeration cup. Due to the shape adaptation between the annular shell 304 and the cup body 200, gaps or empty areas caused by shape mismatch are avoided, allowing the power supply component 300 to cleverly integrate into the overall structure and achieve higher integration. In an implementation mode, the cup body 200 includes a first bottom surface 203, and the battery housing 301 includes a second bottom surface 306. The first bottom surface 203 and the second bottom surface 306 jointly support the insulin refrigeration cup, rendering it more stable. On one side of the battery housing 301 away from the cup body 200, there is an anti-slip pad 305, and the anti-slip pad 305 is a vertical plane that provides a more stable surface for the user to apply lateral force to his fingers, preventing the cup from rotating or sliding in his hands, making one handed operation easier and more accurate.

The above embodiments are only used to illustrate the technical solution of the present disclosure and not to limit it. Although the present disclosure has been described in detail with reference to the preferred embodiments, those skilled in the art should understand that the technical solution of the present disclosure can be modified or equivalently replaced without departing from the spirit and scope of the technical solution of the present disclosure.

What is claimed is:

1. An insulin pen-type syringe refrigeration cup, with an insulin pen cartridge provided therein, and the insulin pen-type syringe refrigeration cup comprises:
a refrigeration component, which comprises a semiconductor refrigeration chip, a heat conduction component, and a heat dissipation fan; wherein the semiconductor refrigeration chip comprises a cold end and a hot end, and the hot end is in contact with the heat conduction component,
a cold conduction tube, which comprises a sealed end and an open end, wherein the cold end is in contact with the sealed end, the cold conduction tube comprises an avoidance groove extending from the open end to the sealed end, wherein the cold conduction tube defines a storage space that is configured to accommodate up to one or two insulin pens, and the storage space is further configured to accommodate the insulin pen cartridge, and/or, a diameter of the cold conduction tube is 20mm to 50mm,
a cup lid, configured to accommodate the refrigeration component, wherein the sealed end is fixedly connected to the semiconductor refrigeration chip and/or the cup lid,
a cup body, which comprises a cup wall and an insulation chamber enclosed by the cup wall, wherein the open end extends into the insulation chamber, and a part of the insulin pen or a part of the insulin pen cartridge is configured to extend into the avoidance groove or enter the insulation chamber by passing through the avoidance groove; the cup lid is configured to detachably seal the insulation chamber, and the refrigeration component keeps the insulation chamber at a low temperature.

2. The insulin pen-type syringe refrigeration cup according to claim 1, wherein two insulin pens are configured to be inserted side by side into the cold conduction tube through the open end, or the insulin pen and the insulin pen cartridge are configured to be inserted side by side into the cold conduction tube through the open end.

3. The insulin pen-type syringe refrigeration cup according to claim 2, wherein the cup wall comprises an inner wall, an outer wall, and a hollow cavity provided between the two,
the hollow cavity is in a vacuum state, and there is an insulation gap between the cold conduction tube and the inner wall.

4. The insulin pen-type syringe refrigeration cup according to claim 3, wherein the cold conduction tube comprises two avoidance grooves that are symmetrically arranged.

5. The insulin pen-type syringe refrigeration cup according to claim 4, wherein lengths of the avoidance grooves do not exceed 40% of a length of the cold conduction tube; and/or
widths of the avoidance grooves do not exceed 15% of a perimeter of the cold conduction tube.

6. The insulin pen-type syringe refrigeration cup according to claim 5, wherein the avoidance grooves comprise a main body groove, and an open groove opening and a sealed groove bottom that are provided at two ends of the main body groove,
the open groove opening is provided at the open end, and the sealed groove bottom is in a circular arc structure.

7. The insulin pen-type syringe refrigeration cup according to claim 1, wherein the insulin pen-type syringe refrigeration cup further comprises a power supply component configured to supply energy to the refrigeration component;
the cup lid is provided with a first chamber and a second chamber that are arranged in parallel;
the first chamber is configured to accommodate the refrigeration component, and the second chamber is configured to accommodate a refrigeration control circuit board;
the power supply component is arranged below the second chamber, and the refrigeration control circuit board is electrically connected to the refrigeration component and the power supply component, respectively.

8. The insulin pen-type syringe refrigeration cup according to claim 7, wherein the semiconductor refrigeration chip is horizontally arranged in the first chamber and separates the first chamber to form a heat conduction chamber and a cold conduction chamber,
the heat conduction chamber is configured to accommodate the heat conduction fan and the heat conduction component,
the cup lid is provided with an air inlet and an air outlet that are configured to communicate the heat conduction chamber,
the sealed end is configured to extend into the cold conduction chamber, and the cup body extends into the cold conduction chamber and is detachably connected to the cup lid.

9. The insulin pen-type syringe refrigeration cup according to claim 8, wherein the heat dissipation fan comprises a fan body and a fan frame configured to install the fan body, and the fan frame is clamped to the cup lid.

10. The insulin pen-type syringe refrigeration cup according to claim 7, wherein the power supply component comprises a battery housing, the battery housing comprises an annular shell that matches a shape of the cup body, and the power supply component and the cup body jointly support the insulin pen-type syringe refrigeration cup.
